(19) United States
(12) Patent Application Publication
(10) Pub. No.: US 2007/0100179 A1
(43) Pub. Date: May 3, 2007

(54) Novel compounds for an organic electroluminescent device and an organic electroluminescent device comprising said compounds

(76) Inventors: Chun-Liang Lai, Jiayi Hsien (TW); Kuo-Wei Huang, Xinzhuang City (TW); Ching-Hung Chen, Taichung (TW)

Correspondence Address: HERSHKOVITZ & ASSOCIATES, 2845 DUKE STREET, ALEXANDRIA, VA, 22314, US

(21) Appl. No.: 11/260,178

(22) Filed: Oct. 28, 2005

(51) Int. Cl. C07C 13/567; C09K 11/06; H01L 51/54

(52) U.S. Cl. 585/26; 428/690; 428/917; 585/27; 313/504; 313/506; 257/E51.049; 257/E51.051; 257/102; 257/103

(57) ABSTRACT

The present invention relates to compounds represented by formula (1) 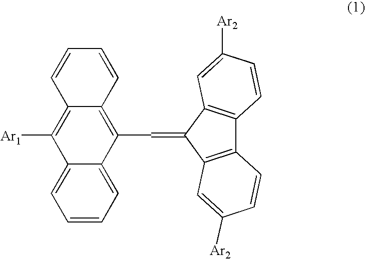
wherein Ar₁ is a substituted or unsubstituted C₆-C₁₈ aryl group, and Ar₂ is hydrogen or a substituted or unsubstituted C₆-C₁₂ aryl group; and by formula (2) 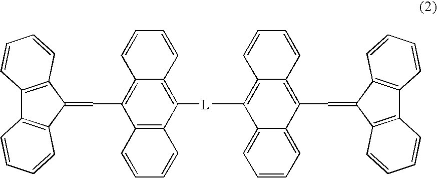
wherein L is a substituted or unsubstituted C₆-C₁₄ aryl group. The present invention also relates to an organic electroluminescent device comprising said compounds. The organic electroluminescent device according to the invention has advantages of high luminous efficiency, high color purity, high thermal stability and a long operational life.

### FIELD OF THE INVENTION

The present invention relates to compounds for an organic electroluminescent (EL) device and to an organic EL device comprising said compounds. The organic EL device according to the invention has high luminous efficiency, high color purity, high thermal stability and a long operational life.

### BACKGROUND OF THE INVENTION

An organic EL device is typically composed of an anode, a cathode and layers of organic materials disposed between the anode and the cathode. An organic EL device has many excellent properties such as self-light emitting, low thickness, wide angle of view, quick response, etc. Therefore, organic EL devices will be the basis of the next generation of displays. However, a significant deficiency that greatly restricts the applications of organic EL devices and needs to be addressed immediately is its short lifetime.

There are many factors leading to the degradation of an organic EL device, including the crystallization of organic materials, the thermal instability of organic materials and the poor efficiency of energy conversion between luminescent materials. The latter results in the degradation of the host- and dopant-materials in the luminescent layer.

Efforts have been made to improve the efficiency of the energy conversion between the luminescent materials. However, the lifetime of an organic EL device has not been increased to an extent that meets a practical standard. For example, U.S. Pat. Nos. 5,935,721 and 5,972,247 disclose anthracene derivatives for use as the luminescent materials in an organic EL device. However, the organic EL device has poor luminous efficiency and therefore is not practical.

EP 1 215 739 discloses a tetraphenylmethane derivative for use as a blue light emitting material. However, the derivative has a large molecular weight and a poor utilization ratio in the preparation of an organic EL device. Therefore the tetraphenylmethane derivative is also not practical.

JP2003146951 discloses an anthracene derivative for use as a luminescent material in an organic EL device. The organic EL device obtained has a short lifetime and therefore is not practical.

JP2004075580 discloses a dianthracene derivative for use as a luminescent material in an organic EL device. While the color purity of the organic EL device is improved efficiently, the preparation of the luminescent material is difficult, and the practical utility of the luminescent material is difficult to be achieved.

To increase the operational life of an organic EL device, inventors of the present invention conducted extensive experiments and finally found that an anthracene-fluorene based derivative has high thermal stability and is suitable for use in combination with a diarylamino-substituted compound of high luminous efficiency in the luminescent layer of an organic EL device. The organic EL device according to the invention satisfies the demands for high luminous efficiency, high color purity and a long operational life.

### SUMMARY OF THE INVENTION

An objective of the invention is to provide novel compounds represented by following formulae (1) and (2), 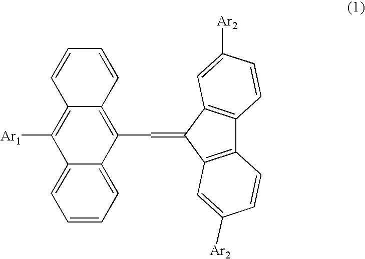
wherein Ar₁ is a substituted or unsubstituted C₆-C₁₈ aryl group, and Ar₂ is hydrogen or a substituted or unsubstituted C₆-C₁₂ aryl group; 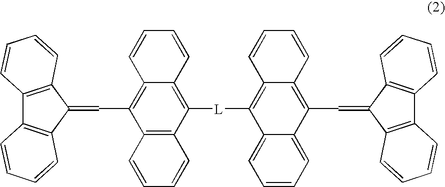
wherein L is a substituted or unsubstituted C₆-C₁₄ aryl group.

Another objective of the invention is to provide an organic EL device comprising an EL compound represented by formula (I) or (2).

An organic EL device according to the present invention having high luminous efficiency, high color purity and a long operational life comprising a compound of formula (1) or (2) comprises an anode, a cathode and layers of organic compound films between the anode and the cathode. The organic compound films include at least one luminescent layer that is formed from the compound of formula (1) or (2) doped with a diarylamino-substituted compound of high luminous efficiency.

### DETAILED DESCRIPTION OF THE INVENTION

A compound according to the invention for use as a luminescent material in a luminescent layer of an organic EL device is represented by formula (1), 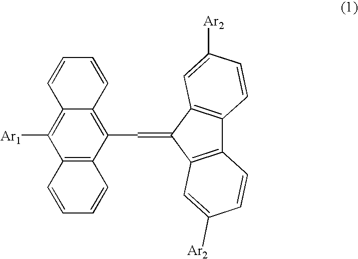
wherein Ar₁ is a substituted or unsubstituted C₆-C₁₈ aryl group, and Ar₂ is hydrogen or a substituted or unsubstituted C₆-C₁₂ aryl group.

In preferred compounds of formula (1), Ar₁ is a phenyl which is unsubstituted or substituted by one or more linear or branched C₁-C₄ alkyl or C₁-C₄ alkoxy groups, preferably the number of the substituting groups is no more than three; biphenyl, preferably o-biphenyl or m-biphenyl; 1-naphthyl which is unsubstituted or substituted by one or more linear or branched C₁-C₄ alkyl or C₁-C₄ alkoxy groups; 2-naphthyl which is unsubstituted or substituted by one or more linear or branched C₁-C₄ alkyl or C₁-C₄ alkoxy groups; 9-phenanthryl; or 1-pyrenyl; and

Ar₂ is a phenyl which is unsubstituted or substituted by one or more linear or branched C₁-C₄ alkyl or C₁-C₄ alkoxy groups, preferably the number of the substituting groups is no more than three; biphenyl, preferably o-biphenyl or m-biphenyl; 1-naphthyl which is unsubstituted or substituted by one or more linear or branched C₁-C₄ alkyl or C₁-C₄ alkoxy groups; or 2-naphthyl which is unsubstituted or substituted by one or more linear or branched C₁-C₄ alkyl or C₁-C₄ alkoxy groups.

More preferably, the compound of formula (1) is, for example, one of the following compounds H1-H9:

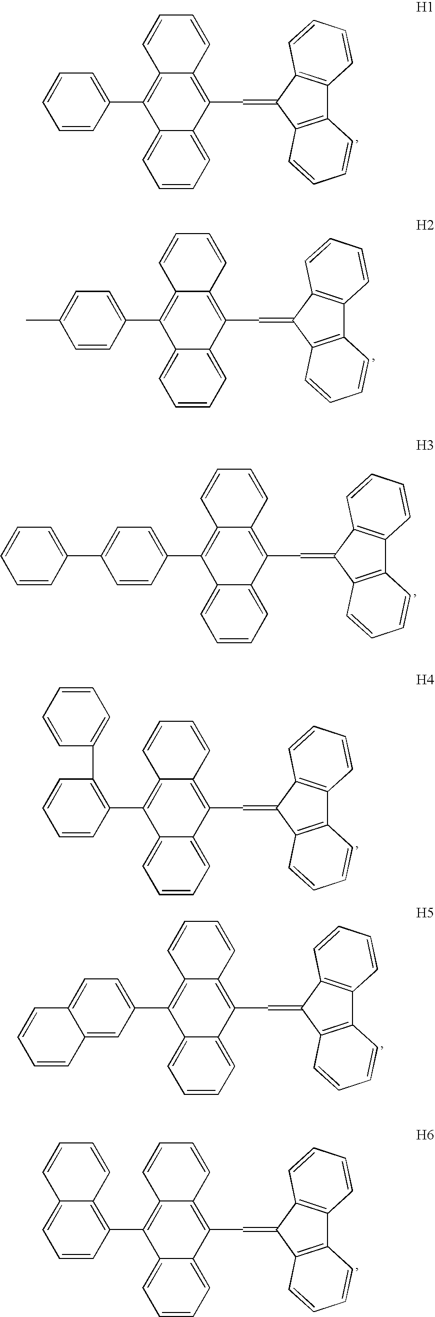

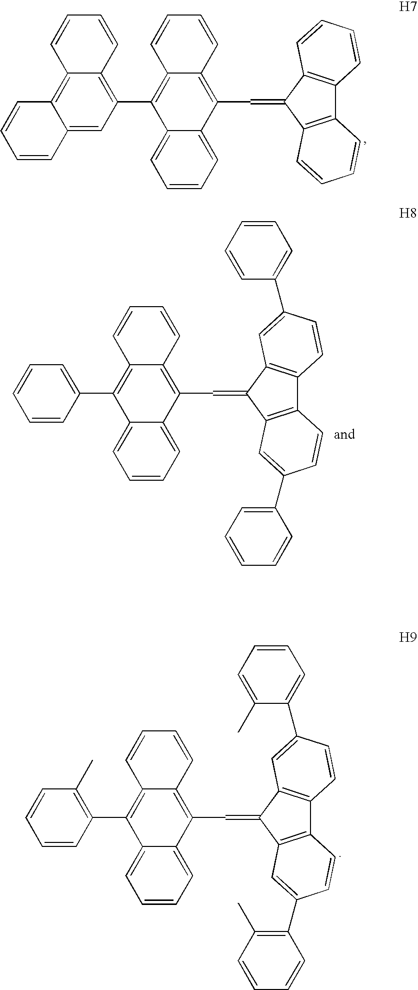

A compound according to the invention for use as a luminescent material in a luminescent layer of an organic EL device is represented by formula (2), 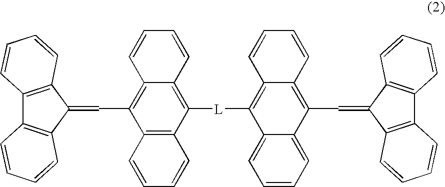
wherein L is a substituted or unsubstituted C₆-C₁₄ aryl group.

In preferred compounds of formula (2), L may be a radical represented by one of the following formulae:

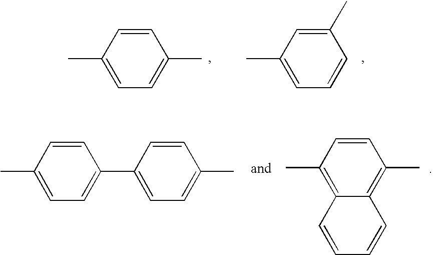

More preferably, the compound of formula (2) is, for example, one of the following compounds H10-H12:

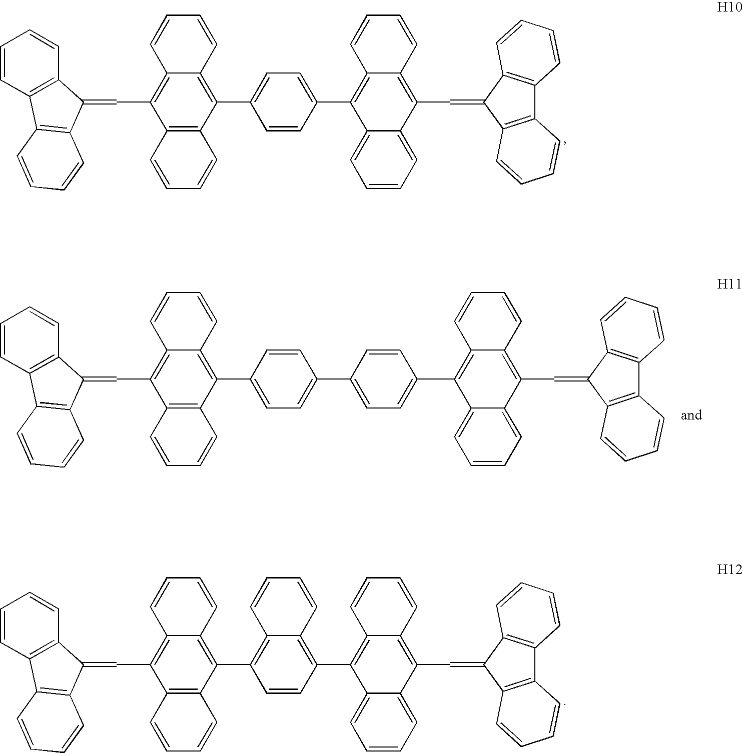

The compound of formula (1) or (2) can be prepared, for example, with the procedure depicted below:

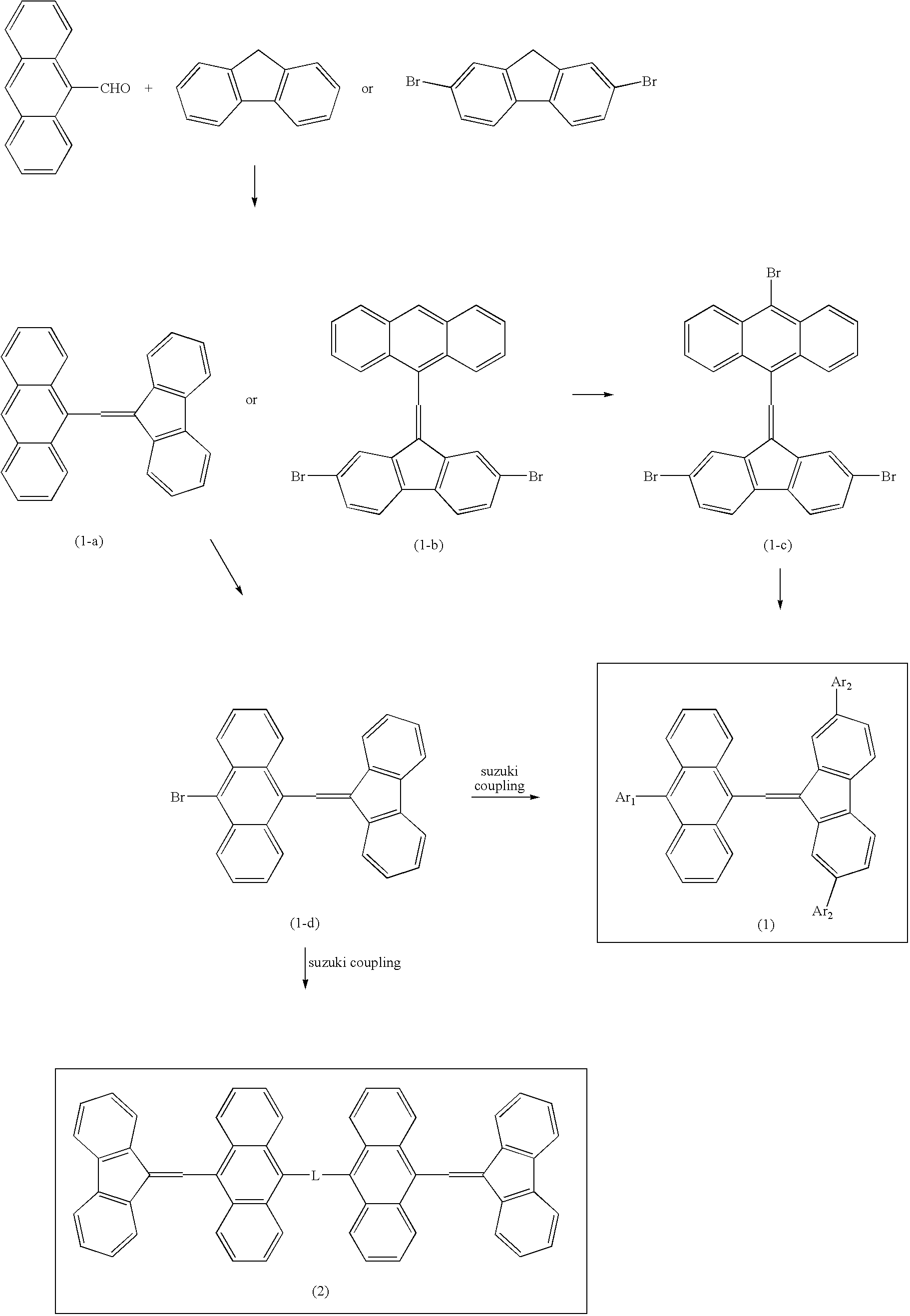

As shown above, a reaction of 9-anthraldehyde and fluorene under an alkaline condition results in intermediate (1-a). Bromination of intermediate (1-a) leads to intermediate (1-d). Alternatively, coupling of 9-anthraldehyde with 2,7-dibromofluorene under an alkaline condition results in intermediate (1-b). Bromination of intermediate (1-b) leads to intermediate (1-c). The compound of formula (I) can be obtained from a subsequent Suzuki coupling of intermediate (1-d) or (1-c). The compound of formula (2) can be obtained from a subsequent Suzuki coupling of intermediate (1-d).

The compounds of formula (1) and (2) obtained can be purified by column chromatography, recrystallization or sublimation, and the purity of the compounds can be above 99%. Preferably, sublimation is employed for purification of the compounds since sublimation has the advantages of: 1) effectively removing mineral salts; 2) improving the particle compactness of the product; and 3) assuring completely drying the product to reduce factors causing degradation of an organic EL device.

By way of example, an organic EL device may comprise, in sequence, an anode, a hole-injecting layer, a hole-transporting layer, a luminescent layer, an electron-transporting layer, an electron-injecting layer and a cathode. Alternatively, an organic EL device may comprise, in sequence, an anode, a hole-transporting layer, a luminescent layer, an electron-transporting layer, an electron-injecting layer and a cathode. Further, an organic EL device may comprise, in sequence, an anode, a hole-transporting layer, a luminescent layer, an electron-transporting layer and a cathode.

Typically, the manufacture of an organic EL device uses substrate of a transparent material such as glass. Organic materials used in the formation of an organic EL device are heated in a vacuum (<10⁻³ torr) to 200˜600° C. for being directly vaporized in fabrication equipment and then subsequently are deposited on the substrate to form films. The fabrication equipment uses a quartz vibrator to control the thickness of the films.

Typically, the anode is made of a metal, an alloy or a conductive compound having a work function higher than 4 eV, e.g., indium-tin-oxide (ITO), gold or the like. The anode preferably has a resistivity of less than 100 Ω/□ and a thickness of 50˜200 nm.

The cathode is made of a metal, an alloy or the like and has a work function lower than 4 eV, e.g., Al, Li, Mg, Ag, Al—Li alloy, Mg—Ag alloy or the like. The cathode preferably has a thickness of 50˜200 nm.

The electron-injecting layer mainly consists of a metal or an inorganic ionic compound, such as LiF, Cs or the like. The electron-injecting layer preferably has a thickness of less than 1 nm.

The hole-injecting layer may be made from conventional phthalocyanine dyes, such as copper phthalocyanine and zinc phthalocyanine, or triarylamine derivatives, such as m-TDATA (4,4′,4″-tris(N-3-methyl-phenyl-N-phenyl-amino)triphenylamine) and 1-TNATA (4,4′,4″-tris(N-(1-naphthyl)-N-phenyl-amino)triphenylamine). The hole-injecting layer preferably has a thickness of 20˜80 nm.

The hole-transporting layer may be formed from conventional NPB (N,N′-di(naphthalene-1-yl)-N,N′-diphenyl-benzidine), PPB (N,N′-bis(phenanthien-9-yl)-N,N′-diphenylbenzidine) or spiro-TAD (2,2′,7,7′-tetra-(diphenylamino)-9,9′-spiro-bifluorene). The hole-transporting layer preferably has a thickness of 10˜50 nm.

The luminescent layer is composed of a host material and a dopant having high luminous efficiency. When a current passes through the organic EL device, an electron and a hole recombine in the luminescent layer and the host material is excited and generates a photon. Then, energy is transferred to the dopant and the dopant is excited. When the dopant returns to the base state, the energy is released in the form of light. With the incorporation of a dopant into the host material, the energy can be utilized efficiently and will not transform to heat. Therefore, the luminous efficiency of the organic EL device is superior to that employing a single luminescent material.

The color of light of an organic EL device depends on the dopant used. Thus, a blue dopant of high luminous efficiency should be used if blue light is to be emitted. This principle applies analogously to other colors.

The compound of formula (1) or (2) according to the invention is mainly used as the host luminescent material of an organic EL device. The highest occupied molecular orbital (HOMO) of the compound of formula (1) or (2) according to the invention is 5.5˜6.0 eV. Suitable dopants for combination with the compound according to the invention include but are not limited to, for example, diarylamino-substituted biphenyl derivatives, diarylamino-substituted binaphthalene derivatives, diarylamino-substituted anthracene derivatives, diarylamino-substituted bianthracene derivatives, diarylamino-substituted stilbene derivatives, 6,6′-bis(diarylamino-substituted) 2,2′-trans-naphthylene vinylene naphthylene derivatives, diarylamino-substituted fluorene derivatives, diarylamino-substituted pyrene derivatives, coumarin derivatives, rubrene derivatives, pentacene derivatives, polyarylhydrocarbon derivatives and the like.

An example of manufacturing a blue light emitting device has a luminescent layer preferably formed from a compound of formula (1) or (2) in combination with a 6,6′-bis(diarylamino-substituted) 2,2′-trans-naphthylene vinylene naphthylene derivative. The 6,6′-bis(diarylamino-substituted) 2,2′-trans-naphthylene vinylene naphthylene derivative is represented by formula (3) below: 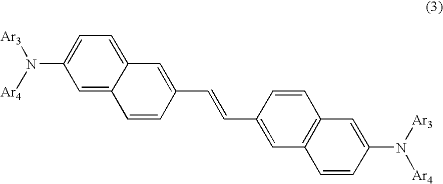
wherein Ar₃ and Ar₄ may be identical or different and are independently a substituted or unsubstituted C₆-C₁₅ aryl group.

In the preferred compounds of formula (3), Ar₃ and Ar₄ are independently phenyl, p-tolyl, m-tolyl, o-tolyl, p-biphenyl, o-biphenyl, 1-naphthyl, 2-naphthyl, 9-phenanthryl and the like.

The compound of formula (3) is, for example, one of the compounds of formulae D1˜D4:

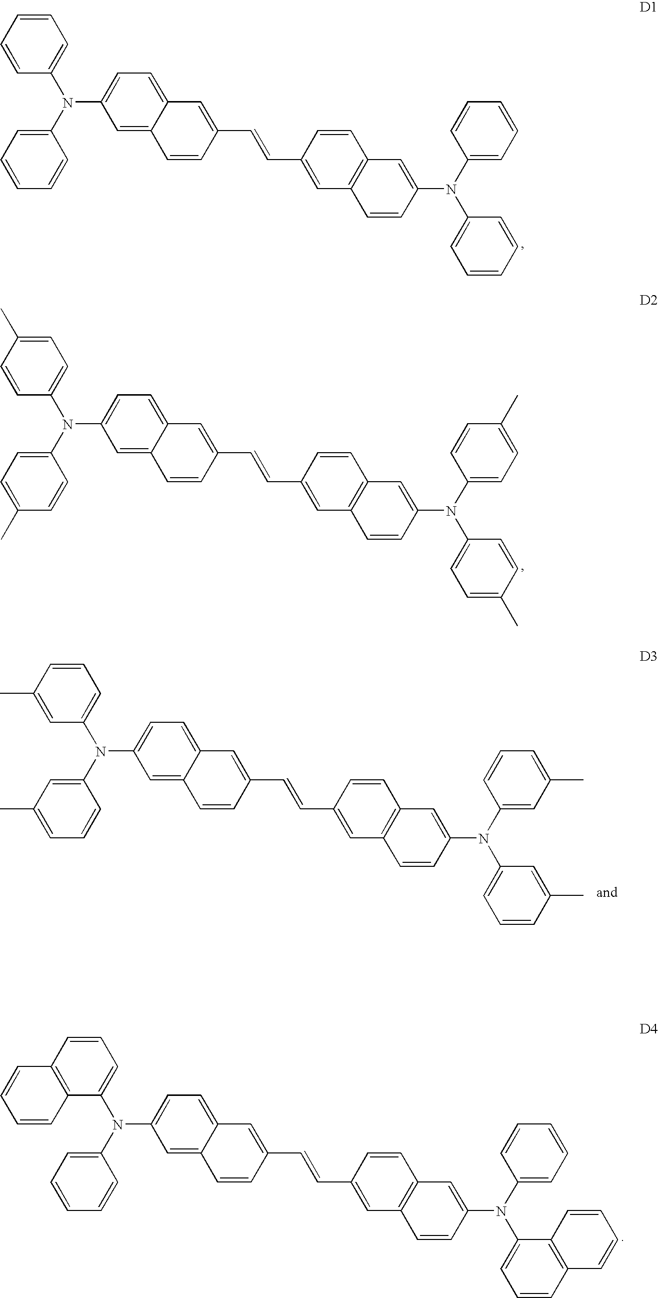

Generally, a dopant is preferably present in an amount of 0.5˜10% by weight of the host material. The luminescent layer preferably has a thickness of 10˜50 nm.

The electron-transporting layer of an organic EL device may be formed from a metal-quinolinate complex, such as Alq₃ (tris(8-hydroxyquinolinato)aluminum), Bebq₂ (bis(10-hydroxybenzo[h]quinolinato)beryllium), Gaq₃ (tris(8-hydroxyquinolinato)gallium) and the like, a triazine derivative, or an oxadiazole derivative. The metal-quinolinate complex is a commonly used electron-transporting material since it has high thermal stability and can be directly vaporized in a vacuum and at elevated temperatures. The electron-transporting layer preferably has a thickness of 10˜50 nm.

An example of the fabrication of a preferred embodiment of the organic EL device according to the invention follows.

An anode is formed by deposition or sputtering of anode material by vacuum evaporation onto a suitable transparent substrate. Next, an electron-injecting layer, a hole-transporting layer, a luminescent layer, an electron-transporting layer and an electron-injecting layer are formed in sequence by deposition by vacuum evaporation. Generally, the vacuum is below 10⁻³ torr, and the rate of deposition is preferably 0.01˜5 nm/s. Finally, a cathode is formed by deposition or sputtering by vacuum evaporation to complete the organic EL device. The organic EL device may be suitably packaged and then can be operated in atmosphere.

Alternatively, the organic EL device may be fabricated in a reversed sequence. Specifically, a cathode is first formed on the substrate and an electron-injecting layer, an electron-transporting layer, a luminescent layer, a hole-transporting layer and a hole-injecting layer are formed in sequence, and finally an anode is formed. When a direct current is applied, the organic EL device will emit light stably and continuously.

The following examples further illustrate the invention.

### EXAMPLES

### Example 1

### Synthesis of Compound H3

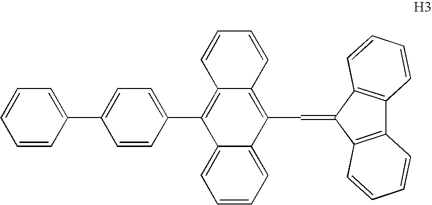 a) Synthesis of Intermediate (1-a)

20 g of fluorene, 27.3 g of 9-anthraldehyde, 14.4 g of sodium hydroxide, 3.7 g of tetramethylammonium bromide, 120 mL of toluene and 60 mL of water were mixed in a reaction vessel and heated to 80° C. under a nitrogen blanket. The reaction mixture was stirred for 10 hours. Thereafter, the aqueous layer was drawn off while hot, and 100 mL of methanol was added. A solid was precipitated. The solid was filtered off and dried at 100° C. to yield 37 g (yield 88%) of intermediate (1-a) as a yellow solid. m.p.=238° C.

b) Synthesis of Intermediate (1-d)

20 g of intermediate (1-a) and 188 mL of methylene chloride were mixed in a reaction vessel. 4.35 mL of Bromine was then added at room temperature under a nitrogen blanket. The reaction mixture was stirred for 30 minutes, and then 200 mL of methanol was added. The precipitate was filtered off and dried at 100° C. to yield 19.5 g (yield 80%) of intermediate (1-d) as a yellow solid. ¹H NMR (CDCl₃, 200 MHz): δ 8.60 (d, 2H), 8.16 (d, 2H), 8.09 (s, 1H), 8.02 (m, 1H), 7.66(m, 1H), 7.55˜7.63 (m, 3H), 7.34˜7.46 (m, 4H), 7.13 (t, 1H), 6.60 (t, 1H), 6.05(d, 1H).

c) Synthesis of Compound H3

10 g of intermediate (1-d), 5.48 g of biphenyl-4-boronic acid, 5.92 g of tripotassium phosphate, 10 mg of palladium acetate, 19 mg of tris(tert-butyl)phosphine and 38.5 mL of xylene were mixed in a reaction vessel and heated to 140° C. under a nitrogen blanket. The reaction mixture was stirred for 3 hours, and then 70 mL of toluene was added. The mixture was filtered while hot. Next, the filtrate was concentrated by evaporation, and the residue was dried to yield a yellow solid. The solid was purified by sublimation to yield 9.7 g (yield 83%) of the compound H3. Tg=121° C. and Tm=308° C.

### Example 2

### Synthesis of Compound H5

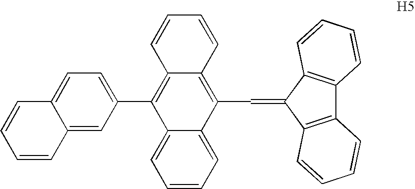

10 g of intermediate (1-d), 4.76 g of naphthyl-2-boronic acid, 7.35 g of tripotassium phosphate, 15 mg of palladium acetate, 28 mg of tris(tert-butyl)phosphine and 38.5 mL of xylene were mixed in a reaction vessel and heated to 140° C. under a nitrogen blanket. The reaction mixture was stirred for 3 hours, and then 70 mL of toluene was added. The mixture was filtered while hot. Next, the filtrate was concentrated by evaporation, and the residue was dried to yield a yellow solid. The solid was purified by sublimation to yield 9.8 g (yield 72%) of compound H5. Tg=137.6° C.; Tm=333.3° C.; ¹H NMR (CDCl₃, 400 MHz): δ 8.28 (t, 3H), 8.13˜8.08 (m, 3H), 8.05˜8.03 (m, 1H), 7.97˜7.90 (m, 1H), 7.83˜7.76 (m, 4H), 7.74˜7.69 (m, 1H), 7.65˜7.58 (m, 2H), 7.51˜7.44 (m, 2H), 7.40˜7.31 (m, 4H), 7.21˜7.17 (m, 1H), 6.71 (q, 1H), 6.32 (t, 1H).

### Example 3

### Synthesis of Compound H8

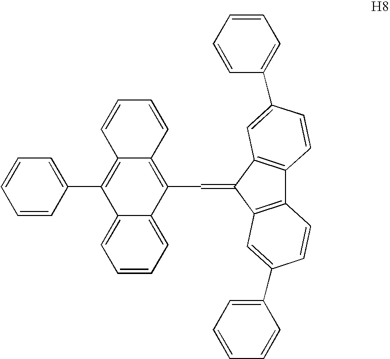 a) Synthesis of Intermediate (1-b)

50 g of 2,7-dibromofluorene, 35 g of 9-anthraldehyde, 18.5 g of sodium hydroxide, 2.38 g of tetramethylammonium bromide, 154 mL of toluene and 77 mL of water were mixed in a reaction vessel and heated to 80° C. under a nitrogen blanket. The reaction mixture was stirred for 10 hours. Next, the aqueous layer was drawn off while hot, and 200 mL of methanol was added. The mixture was filtered, and the solid obtained was dried at 100° C. to yield 42.7 g (yield 54%) of intermediate (1-b) as a yellow solid, which was used in the next step without further purification. 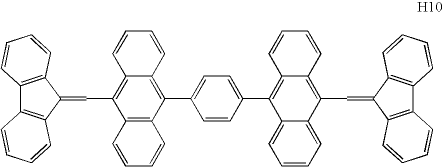
b) Synthesis of Intermediate (1-c)

20 g of intermediate (1-b) and 130 mL of methylene chloride were mixed in a reaction vessel, and then 3 mL of bromine was added dropwise at room temperature under a nitrogen blanket. The reaction mixture was stirred for 30 minutes, and 200 mL of methanol was added. A solid was precipitated. The solid was filtered off and dried at 100° C. to yield 19.0 g (yield 83%) of intermediate (1-c) as a yellowish green solid, which was used in the next step without further purification.

c) Synthesis of Compound H8

10 g of intermediate (1-c), 7.43 g of phenylboronic acid, 12.6 g of tripotassium phosphate, 11 mg of palladium acetate, 21 mg of tris(tert-butyl)phosphine and 33.8 mL of xylene were mixed in a reaction vessel and heated to 140° C. under a nitrogen blanket. The reaction mixture was stirred for 3 hours, and then 150 mL of toluene was added. The mixture was filtered while hot at 100° C. Next, the filtrate was concentrated by evaporation, and the residue was dried to yield a yellow solid. The solid was purified by sublimation to yield 7.2 g (yield 72%) of compound H8. Tg=117° C. and Tm=302° C.

### Example 4

### Synthesis of Compound H10

31.37 g of intermediate (1-d), 5 g of phenyl-1,4-diboronic acid, 14.49 g of tripotassium phosphate, 27 mg of palladium acetate, 49 mg of tris(tert-butyl)phosphine and 60 mL of xylene were mixed in a reaction vessel and heated to 140° C. under a nitrogen blanket. The reaction mixture was stirred for 3 hours, and then 240 mL of toluene was added. The mixture was filtered while hot at 100° C. Next, the filtrate was concentrated by evaporation, and the residue was dried to yield a yellow solid. The solid was purified by sublimation to yield 14.1 g (yield 59%) of compound H10. Tg=202° C. and Tm=427° C.

### Example 5

### Fabrication of an Organic EL Device (1) According to the Invention

An ITO glass substrate with a surface resistivity of 20 Ω/□ was placed in a vacuum vessel of a vapor deposition machine. A crucible containing 2-TNATA, a crucible containing NPB, a crucible containing compound H3 of the present invention, a crucible containing compound D2, a crucible containing tris(8-hydroxylquinolinato)aluminum (Alq₃), a crucible containing aluminum and a crucible containing lithium fluoride were placed in the machine.

The pressure in the vacuum vessel on the machine was reduced to 10⁻⁶ torr. The crucible containing 2-TNATA was heated and 2-TNATA was deposited on the glass substrate by evaporation at a rate of 0.2 nm/s to form a hole-injecting layer having a thickness of 60 nm. Next, a NPB film having a thickness of 20 nm was formed on the hole-injecting layer as a hole-transporting layer at a rate of 0.2 nm/s from the crucible containing NPB. Subsequently, the crucibles containing compound H3 and compound D2 are heated, and a luminescent layer composed of compound H3 incorporated with 4% of compound D2 was formed on the hole-transporting layer at a rate of 0.2 nm/s. The thickness of the luminescent layer is 30 nm. Then, an Alq₃ film having a thickness of 25 mm was formed on the luminescent layer as an electron-transporting layer from the crucible containing Alq₃. Thereafter, a lithium fluoride film having a thickness of 0.7 nm was formed on the electron-transporting layer as an electron-injecting layer by evaporation deposition from the crucible containing lithium fluoride. Finally, an aluminum cathode film having a thickness of 150 nm was formed on the electron-injecting layer from the crucible containing aluminum.

When a direct current of 10 mA/cm² at a potential of 5.4 V was applied to the organic EL device obtained, a blue light was emitted with a light intensity of 720 cd/m² and a CIE coordinate of x=0.15, y=0.15. When the light intensity was set at 500 cd/m², the brightness decreased by half after 1100 hours of operation.

### Example 6

### Fabrication of an Organic EL Device (2) According, to the Invention

The procedure was as the procedure described in Example 5 except that the luminescent layer was composed of compound H5 incorporated with 4% of compound D2. When a direct current of 10 mA/cm² at a potential of 5.4 V was applied to the organic EL device obtained, a blue light was emitted with a light intensity of 710 cd/m² and a CIE coordinate of x=0.15, y=0.15. When the light intensity was set at 500 cd/m², the brightness decreased by half after 1200 hours of operation.

### Example 7

### Fabrication of an Organic EL Device (3) According to the Invention

The procedure was the same as the procedure described in Example 5 except that the luminescent layer was composed of compound H10 incorporated with 4% of compound D2. When a direct current of 10 mA/cm² at a potential of 5.4 V was applied to the organic EL device obtained, a blue light was emitted with a light intensity of 750 cd/m² and a CIE coordinate of x=0.15, y=0.16. When the light intensity was set at 500 cd/m², the brightness decreased by half after 1500 hours of operation.

### Example 8

### Fabrication of a Control Organic EL Device Using Conventional Organic EL Material for Comparison

The procedure was the same as the procedure described in Example 5 except that the luminescent layer was composed of TBADN (2-tert-butyl-9,10-di-2-naphthylanthracene) incorporated with 3% of compound D2. TBADN has a chemical structure shown below.

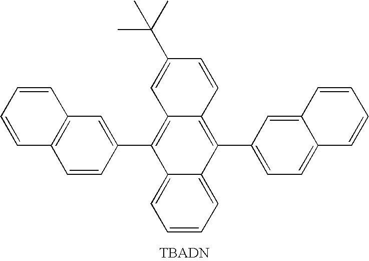

When a direct current of 10 mA/cm² at a potential of 5.4 V was applied to the organic EL device obtained, a blue light was emitted with a light intensity of 510 cd/m² and a CIE coordinate of x=0.15, y=0.16. When the light intensity was set at 500 cd/m², the brightness decreased by half after 700 hours of operation.

The results of Examples 5 to 8 are listed in Table 1.

TABLE 1
Comparison of Organic EL Devices

| | | | Time to 50% |
|---|---|---|---|
| Organic EL | Light Intensity | | Brightness |
| Device | (cd/m²) | CIE Coordinate | (hrs) |
| (1) | 720 | x = 0.15, y = 0.15 | 1100 |
| (2) | 710 | x = 0.15, y = 0.15 | 1200 |
| (3) | 750 | x = 0.15, y = 0.16 | 1500 |
| Control | 510 | x = 0.15, y = 0.16 | 700 |

Table 1 shows that the luminous efficiency and operational life of the organic EL devices are significantly improved with a comparably high color purity when a compound of formula (1) or (2) according to the invention was used as the luminescent material in an organic EL device.

### INDUSTRIAL APPLICABILITY

When a compound of formula (1) or (2) according to the invention is used in combination with a diarylamino-substituted compound to form the luminescent layer in an organic EL device, the device obtained has advantages of high luminous efficiency, high color purity and a long operational life. Such an organic EL device can be advantageously used in display panels of MP3s, digital cameras, cellular telephones, etc.

1. A compound of formula (1), 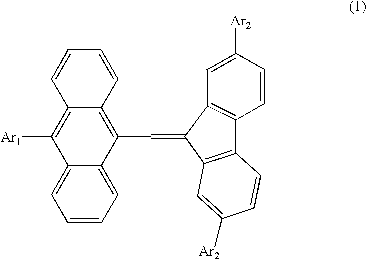
wherein Ar₁ is a substituted or unsubstituted C₆-C₁₈ aryl group, and Ar₂ is hydrogen or a substituted or unsubstituted C₆-C₁₂ aryl group.

2. A compound according to claim 1, wherein
Ar₁ is a phenyl which is unsubstituted or substituted by one or more linear or branched C₁-C₄ alkyl or C₁-C₄ alkoxy groups; biphenyl; 1-naphthyl which is unsubstituted or substituted by one or more linear or branched C₁-C₄ alkyl or C₁-C₄ alkoxy groups; 2-naphthyl which is unsubstituted or substituted by one or more linear or branched C₁-C₄ alkyl or C₁-C₄ alkoxy groups; 9-phenanthryl or 1-pyrenyl; and
Ar₂ is a phenyl which is unsubstituted or substituted by one or more linear or branched C₁-C₄ alkyl or C₁-C₄ alkoxy groups; biphenyl; 1-naphthyl which is unsubstituted or substituted by one or more linear or branched C₁-C₄ alkyl or C₁-C₄ alkoxy groups; or 2-naphthyl which is unsubstituted or substituted by one or more linear or branched C₁-C₄ alkyl or C₁-C₄ alkoxy groups.

3. A compound according to claim 1 selected from the group consisting of:

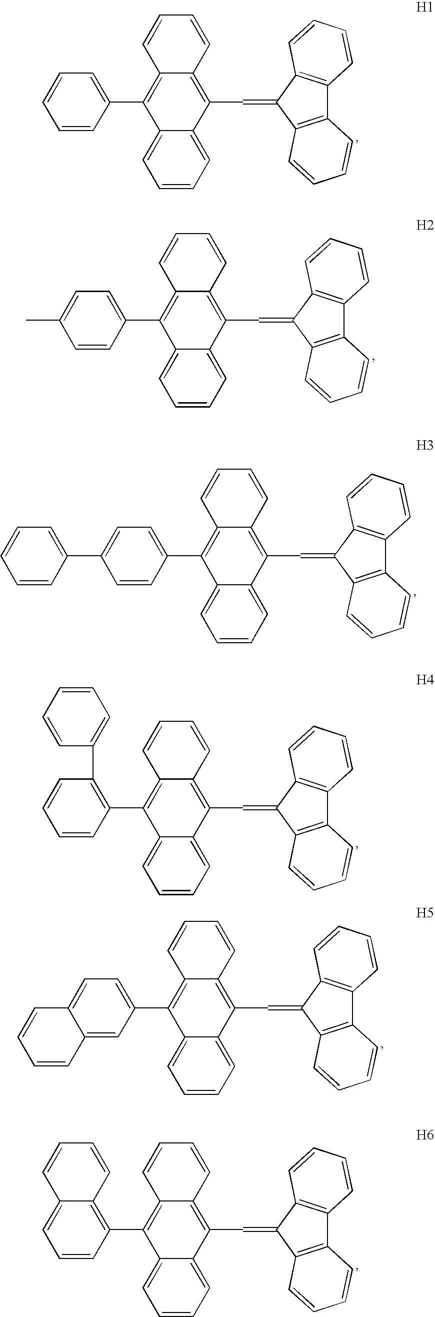

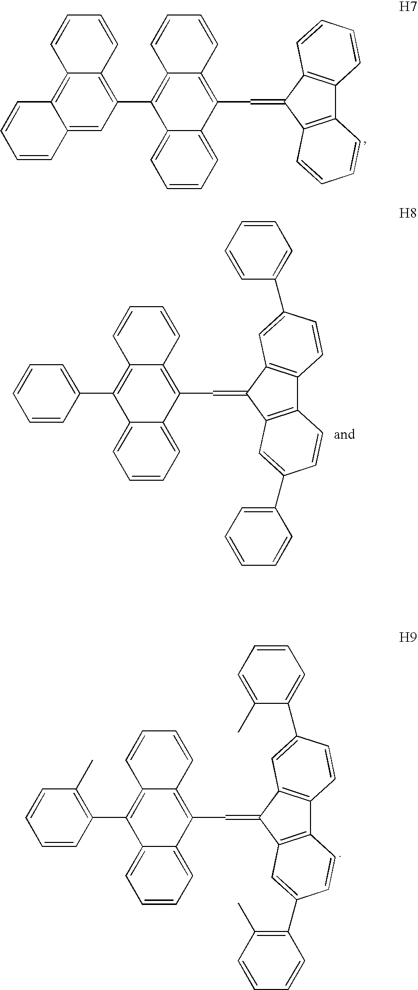

4. A compound according to claim 2. wherein the phenyl has no more than three substituting groups.

5. A compound according to claim 2, wherein the biphenyl is o-biphenyl or m-biphenyl.

6. A compound of formula (2). 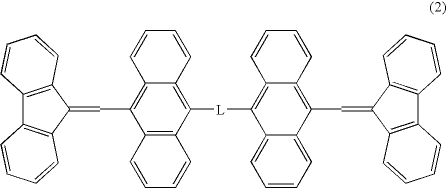
wherein L is a substituted or unsubstituted C₆-C₁₄ aryl group.

7. A compound according to claim 6, wherein L is a radical represented by one of the following formulae:

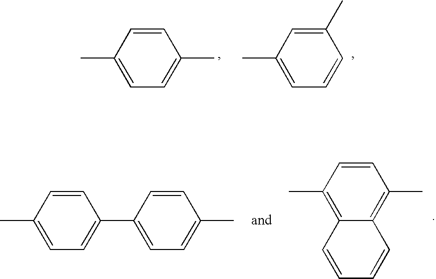

8. A compound according to claim 6 selected from the group consisting of:

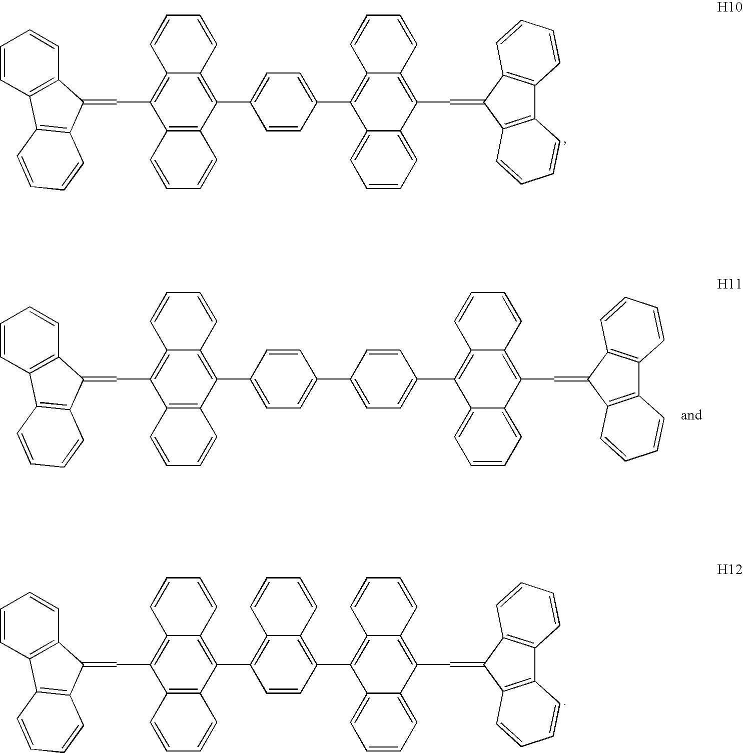

9-13. (canceled)

14. An organic EL device comprising the compound according to claim 1.

15. An organic EL device comprising the compound according to claim 6.

16. An organic EL device comprising an anode, a cathode and layers of organic compound films between the anode and the cathode, wherein the organic compound films include at least one luminescent layer formed from the compound according to claim 1 and a dopant.

17. An organic EL device comprising an anode, a cathode and layers of organic compound films between the anode and the cathode, wherein the organic compound films include at least one luminescent layer formed from the compound according to claim 6 and a dopant.

18. The organic EL device according to claim 11, wherein the dopant is a diarylamino-substituted compound.

19. The organic EL device according to claim 12, wherein the dopant is a diarylamino-substituted compound.
